SEQUENCE LISTING (6 sequences)

SEQ ID NO: 1 - DNA, 20 bp, Artificial Sequence
  Completely sequenced
gtgggccgctctaggcacca

SEQ ID NO: 2 - DNA, 22 bp, Artificial Sequence
  Completely synthesized
cggttggccttagggttcaggg

SEQ ID NO: 3 - DNA, 18 bp, Artificial Sequence
  Completely synthesized
tgcccgaaggctacgtcc

SEQ ID NO: 4 - DNA, 17 bp, Artificial Sequence
  Completely synthesized
gcacgctgccgtcctcg

SEQ ID NO: 5 - DNA, 20 bp, Artificial Sequence
  Completely synthesized
atggtgagcaagggcgagga

SEQ ID NO: 6 - DNA, 20 bp, Artificial Sequence
  Completely synthesized
ttgtacagctcgtccatccg

(19) United States
(12) Patent Application Publication
(10) Pub. No.: US 2014/0093964 A1
(43) Pub. Date: Apr. 3, 2014

(54) DELIVERY OF BIOLOGICAL MATERIALS INTO CELLULAR ORGANELLES

(71) Applicant: Brigham Young University, Provo, UT (US)

(72) Inventors: Quentin T. Aten, Orem, UT (US); Sandra H. Burnett, Saratoga Springs, UT (US); Brian D. Jensen, Orem, UT (US); Larry L. Howell, Orem, UT (US)

(73) Assignee: Brigham Young University, Provo, UT (US)

(21) Appl. No.: 14/042,530

(22) Filed: Sep. 30, 2013

Related U.S. Application Data
(62) Division of application No. 13/456,856, filed on Apr. 26, 2012, now abandoned.
(60) Provisional application No. 61/479,777, filed on Apr. 27, 2011.
(60) Provisional application No. 61/536,889, filed on Sep. 20, 2011.

(51) Int. Cl. C12N 15/85

(52) U.S. Cl. CPC C12N 15/85
USPC 435/455; 435/285.2

(57) ABSTRACT

Systems, devices, and methods for delivering a biological material into an organelle of a cell are provided. In one aspect, for example, a method for introducing biological material into an organelle of a cell includes bringing into proximity a lance and a preselected biological material outside of a cell and charging the lance with a polarity and a charge sufficient to electrically associate the preselected biological material with a tip portion of the lance. The method also includes penetrating an outer portion of the cell with the lance and directing and inserting the lance into an organelle, discharging the lance to release at least a portion of the biological material into the organelle, and withdrawing the lance from the cell.

### PRIORITY DATA

This application is a continuation of U.S. patent application Ser. No. 13/456,856, filed on Apr. 26, 2012, which claims the benefit of United States Provisional Patent Application Ser. No. 61/479,777, filed on Apr. 27, 2011, which is incorporated herein by reference in its entirety. This application also claims the benefit of U.S. Provisional Patent Application Ser. No. 61/536,889, filed on Sep. 20, 2011, which is incorporated herein by reference in its entirety.

### GOVERNMENT INTEREST

This invention was made with government support under National Science Foundation Grant No. CMMI-0800606 and CMS-0428532. The United States government has certain rights to this invention.

### BACKGROUND OF THE INVENTION

Microinjection of foreign materials into a biological structure such as a living cell can be problematic. Various transfection techniques include the microinjection of foreign genetic material such as DNA into the nucleus of a cell to facilitate the expression of foreign DNA. For example, when a fertilized oocyte (egg) is transfected, cells arising from that oocyte will carry the foreign genetic material. Thus in one application, organisms can be produced that exhibit additional, enhanced, or repressed genetic traits. In some cases, researchers have used microinjections to create strains of mice that carry a foreign genetic construct causing macrophages to auto-fluoresce and undergo cell death when exposed to a certain drugs. Such transgenic mice have since played roles in investigations of macrophage activity during immune responses and macrophage activity during tumor growth.

Prior art microinjectors function in a similar manner to macro-scale syringes: a pressure differential forces a liquid through a needle and into the cell. In some cases a glass needle that has been fire drawn from a capillary tube can be used to pierce the cellular and nuclear membranes of an oocyte. Precise pumps then cause the expulsion of minute amounts of genetic material from the needle and into the cell. Researchers have produced fine microinjection needles made from silicon nitride and silica glass that are smaller than fire drawn capillaries. These finer needles generally also employ macro-scale pumps similar to those used in traditional microinjectors.

Pronuclear microinjection of DNA, for example, traditionally includes injection of liquid containing the DNA into the pronucleus of a cell such as a zygote. Such injections can be challenging processes due to the potential for cell lysis and chromosomal damage. In part, these challenges have motivated the development of various direct and indirect methods of transgenesis, such as viral transfection and embryonic stem cell targeting and injection. In viral transfection, a transgene is inserted into virus particles, which in turn act as carriers, delivering the genetic material to an oocyte or embryo. In embryonic stem-cell mediated transgenesis, a transgene is first targeted in vitro using a ubiquitous gene, such as ROSA, into embryonic stem cells. The transfected embryonic stem cells are then injected into blastocyst stage embryos, resulting in chimeric offspring. These chimeras must be bred to finally obtain germ line transgenic animals. In another example, existing micro-machined or carbon nanotube microelectromechanical systems (MEMS) designed for DNA delivery into tissue cultures have successfully introduced transgenes into cells, but are unsuitable for use in embryos for transgenic animal production. For example, such techniques require cells to grow around stationary needles or require extended periods of time to release bound DNA into the cells. Furthermore, such MEMS techniques do not provide sufficient mechanical displacement to penetrate a zygote's pronucleus.

### SUMMARY OF THE INVENTION

The present disclosure provides systems, devices, and methods for delivering a biological material into an organelle of a cell. In one aspect, for example, a method for introducing biological material into an organelle of a cell includes bringing into proximity a lance and a preselected biological material outside of a cell and charging the lance with a polarity and a charge sufficient to electrically associate the preselected biological material with a tip portion of the lance. The method also includes penetrating an outer portion of the cell with the lance and directing and inserting the lance into an organelle, discharging the lance to release at least a portion of the biological material into the organelle, and withdrawing the lance from the cell.

In another aspect, a method for transfecting a zygote with a biological material is provided. Such a method includes bringing into proximity a lance and a preselected DNA material outside of a zygote and charging the lance with a polarity and a charge sufficient to electrically associate the preselected DNA material with a tip portion of the lance. The method also includes penetrating an outer portion of the zygote with the lance and directing and inserting the lance into a pronucleus of the zygote, discharging the lance to release at least a portion of the DNA material into the pronucleus, and withdrawing the lance from the zygote.

In yet another aspect, a system for introducing biological material into an organelle of a cell is provided. Such a system includes a lance having a working portion operable to enter a cell, where the working portion having a maximum diameter selected to effectively deliver biological material to an organelle while minimizing damage to the cell. The system also includes a charging system electrically coupled to the lance operable to charge and discharge the lance and a lance manipulation system operable to move the lance into and out of an organelle in a reciprocating motion along an elongate axis of the lance that minimizes damage to the cell.

### DEFINITIONS OF TERMS

In describing and claiming the present invention, the following terminology will be used in accordance with the definitions set forth below.

The singular forms “a,” “an,” and, “the” include plural referents unless the context clearly dictates otherwise. Thus, for example, reference to “a support” can include reference to one or more of such supports, and reference to “an oocyte” can include reference to one or more of such oocytes.

As used herein, the term “biological material” can refer to any material that has a biological use and can be delivered into a cell or a cell organelle. As such, “biological material” can refer to materials that may or may not have a biological origin. Thus, such material can include natural and synthetic materials, as well as chemical compounds, dyes, and the like.

As used herein, the term “charged biological material” may be used to refer to any biological material that is capable of being attracted to or associated with an electrically charged structure. Accordingly, the term charged biological material may be used to refer to those molecules having a net charge, as well as those molecules that have a net neutral charge but possess a charge distribution that allows attraction to the structure.

As used herein, the term “uncharged” when used in reference to a lance may be used to refer to the relative level of charge in the lance as compared to a charged biological material. In other words, a lance may be considered to be “uncharged” as long as the amount of charge on the needle structure is insufficient to associate therewith a useable portion of the charged biological material. Naturally, what is a useable portion may vary depending on the intended use of the biological material, and it should be understood that one of ordinary skill in the art would be aware of what a useable portion is given such an intended use. Additionally it should be noted that a lance with no measurable charge would be considered “uncharged” according to the present definition.

As used herein, the term “associate” is used in one aspect to describe biological material that is in electrostatic contact with a structure due to attraction of opposite charges. For example, DNA that has been attracted to a structure by a positive charge is said to be associated or electrically associated with the structure.

As used herein, the term “sample” when used in reference to a sample of a biological material may be used to refer to a portion of biological material that has been purposefully attracted to or associated with the lance. For example, a sample of a biological material such as DNA that is described as being associated with a lance would include DNA that has been purposefully attracted thereto, but would not include DNA that is attracted thereto through the mere exposure of the lance to the environment. One example of DNA that would not be considered to be a “sample” includes airborne DNA fragments that may associate with the lance following exposure to the air.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint without affecting the desired result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### BRIEF DESCRIPTION OF THE DRAWINGS

FIG. 1A shows a schematic representation of a step of the delivery of a biological material into a cellular organelle in accordance with one embodiment of the present disclosure.

FIG. 1B shows a schematic representation of a step of the delivery of a biological material into a cellular organelle in accordance with another embodiment of the present disclosure.

FIG. 1C shows a schematic representation of a step of the delivery of a biological material into a cellular organelle in accordance with another embodiment of the present disclosure.

FIG. 1D shows a schematic representation of a step of the delivery of a biological material into a cellular organelle in accordance with another embodiment of the present disclosure.

FIG. 1E shows a schematic representation of a step of the delivery of a biological material into a cellular organelle in accordance with another embodiment of the present disclosure.

FIG. 1F shows a schematic representation of a step of the delivery of a biological material into a cellular organelle in accordance with another embodiment of the present disclosure.

FIG. 2 shows a system for delivering a biological material into a cellular organelle in accordance with another embodiment of the present disclosure.

FIG. 3 shows a system for delivering a biological material into a cellular organelle in accordance with another embodiment of the present disclosure.

FIG. 4 shows a system for delivering a biological material into a cellular organelle in accordance with another embodiment of the present disclosure.

FIG. 5 shows optical microscopy images of a biological material delivery system in accordance with another embodiment of the present disclosure.

FIG. 6 shows optical microscopy images of a biological material delivery system in accordance with another embodiment of the present disclosure.

FIG. 7 shows a graphical representation of data in accordance with another embodiment of the present invention.

FIG. 8A shows graphical representations of data in accordance with another embodiment of the present invention.

FIG. 8B shows graphical representations of data in accordance with another embodiment of the present invention.

FIG. 8C shows graphical representations of data in accordance with another embodiment of the present invention.

FIG. 9A shows graphical representations of data in accordance with another embodiment of the present invention.

FIG. 9B shows graphical representations of data in accordance with another embodiment of the present invention.

FIG. 10 shows graphical representations of data in accordance with another embodiment of the present invention.

FIG. 11A shows graphical representations of data in accordance with another embodiment of the present invention.

FIG. 11B shows graphical representations of data in accordance with another embodiment of the present invention.

FIG. 11C shows graphical representations of data in accordance with another embodiment of the present invention.

FIG. 11D shows graphical representations of data in accordance with another embodiment of the present invention.

FIG. 12A shows graphical representations of data in accordance with another embodiment of the present invention.

FIG. 12B shows graphical representations of data in accordance with another embodiment of the present invention.

FIG. 12C shows graphical representations of data in accordance with another embodiment of the present invention.

FIG. 12D shows graphical representations of data in accordance with another embodiment of the present invention.

### DETAILED DESCRIPTION

Methods and associated systems for delivering biological material into an organelle within a cell are provided. Using the presently disclosed techniques can facilitate the delivery of biological material directly into the organelle with enhanced results. As one non-limiting example, DNA can be delivered directly into the pronucleus of a zygote, resulting in genomic integration of the DNA with increased embryo survival rates and increased progeny. While not intending to be bound to any scientific theory, such increased survival rates may be the result of reduced cellular damage from the DNA delivery as compared to prior techniques.

Generally, the present methods and systems utilize the electrical association and dissociation of a biological material to a lance or other delivery device as a mechanism for delivering the biological material into a cellular organelle. Because the biological material can be loaded onto the lance and subsequently released via changes in the charge state of the lance, internal microinjection channels are not required for the delivery of the biological material into an organelle. As such, a lance can be smaller in size and can be formed in configurations that may not be possible with prior delivery devices. These delivery devices can have an outer shape and cross-section that is significantly smaller than traditional injection pipettes. Such smaller outer shapes may be less disruptive to cellular structures, and thus may allow delivery of the biological material into an organelle with less cellular damage.

Once a biological material has been electrically associated with a tip portion of the lance, the lance can be inserted through an outer portion of the cell and into an organelle. With a tip portion of the lance located within the organelle, the lance can be discharged to release at least a portion of the biological material. Once the biological material has been delivered into the organelle, the lance can be withdrawn from the cell.

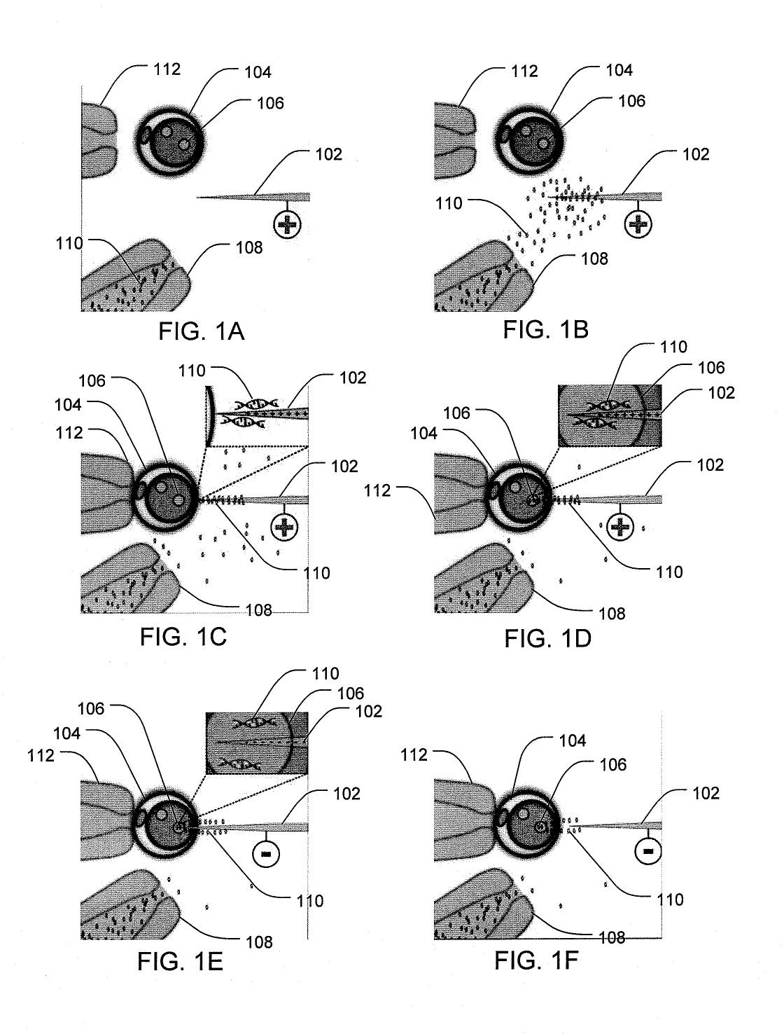

In one aspect, FIGS. 1A-F show exemplary sequences of steps that can be performed to introduce biological material into an organelle of a cell. For this particular example, DNA is used as the biological material, a zygote is used as the cell, and a pronucleus is used as the organelle. This example is intended to be non-limiting, and the description can generally be applied to other biological materials, cells, organelles, and the like. Electrically-mediated delivery of DNA into an organelle can be accomplished due to the unequal charge distributions within DNA molecules. With an effective charge of 2 electrons per base pair, DNA can be manipulated by an electric field. FIG. 1A shows a lance 102 in proximity to a zygote 104 having a pronucleus 106. A biological material delivery device 108 containing the biological material 110 (e.g. DNA in this case), is positioned in proximity to the tip portion of the lance 102. The biological material delivery device 108 is shown as a micropipette, however any device capable of delivery a biological material to the tip portion of the lance is considered to be within the present scope. A cell manipulation device 112 is shown positioned in proximity to the ell 104 to allow manipulation and/or securing of the cell during a biological material delivery procedure.

As is shown in FIG. 1A, the lance 102 is charged with a polarity and a charge sufficient to electrically associate the biological material 110 with a tip portion of the lance 102. In this case, the lance is positively charged in preparation for the accumulation of DNA at a tip portion of the lance. A return electrode is placed in electrical contact with the medium surrounding the lance in order to complete an electrical circuit with the charging device and the lance (not shown). The lance is charged to a degree that is sufficient to associate DNA to the lance during the injection procedure. The amount of voltage sufficient to charge the lance can vary depending on a variety of factors, such as the desired speed of the loading of DNA on the lance, the composition of the lance material, the electrochemical nature of the medium surrounding the lance, and the like.

It should be noted, that various materials begin to decompose (e.g. by electrolysis) at voltages above a certain threshold voltage referred to as the decomposition voltage. The decomposition voltage can be different for different materials. In some cases, such decomposition can generate oxygen and hydrogen at the positively charged lance and the negatively charged return electrode, respectively. These electrolysis products can cause damage to the lance and negatively affect the cell being injected. As such, in one aspect the voltage that can be used to charge the lance can be at or below the decomposition voltage. In one specific aspect, the lance is charged with a voltage from about 1 V below the decomposition voltage to about the decomposition voltage. In another aspect, the lance is charged with a voltage from about 2 V below the decomposition voltage to about the decomposition voltage.

Additionally, voltages higher than the decomposition voltage can cause the biological material to electrophoretically move to the lance. The higher the voltage, the more quickly the biological material will move to and associate with the lance. As such, in some aspects a charging voltage that is higher than the decomposition voltage of the lance can be used. In one aspect, for example, the lance is charged with a voltage from about the decomposition voltage to about 1 V above the decomposition voltage. In another aspect, the lance is charged with a voltage from about the decomposition voltage to about 2 V above the decomposition voltage. In yet another aspect, the lance is charged with a voltage from about the decomposition voltage to about 5 V above the decomposition voltage. In a further aspect, the lance is charged with a voltage that is greater than about 5 V above the decomposition voltage. Additionally, such charging can be described in terms that do not include decomposition voltage. In one aspect, for example, the lance is charged with a voltage from about 0.5 to about 5.0 V. In another aspect, the lance is charged with a voltage from about 1.0 V to about 3 V. In yet another aspect, the lance is charged with a voltage of about 1.5 V.

FIG. 1B shows the biological material 110 being released from the biological material delivery device 108. The biological material 110 can thus be released in the proximity of the tip portion of the lance 102 to effectively allow the biological material to associate with the tip portion of the lance. Thus, in some aspects it can be beneficial to position the biological material delivery device 108 in sufficient proximity to the lance 102 to facilitate this association. It is contemplated that the biological material delivery device can be physically spaced at any distance from the lance; however diffusion of the biological material may occur upon release, thus lowering the effective concentration of the biological material interacting with the tip portion of the lance. In some aspects it can be beneficial to move the lance 102 through the released biological material 110 in order to further facilitating interaction between the two. It should be noted that the lance 102 can be charged before, after, or during introduction of the biological material 110 into the medium surrounding the lance 102. Additionally, in some aspects the lance can be introduced into the medium after the introduction of biological material into the medium.

The polarity of the charge on the lance would depend on the charge distribution of the biological material. In the example shown in FIG. 1B, DNA is the biological material and therefore the lance is charged with a positive polarity to associate the DNA molecules thereto. The positive charge on the lance thus causes the negatively charged DNA to associate with and accumulate at the tip portion of the lance. If a biological material having a positive charge distribution is to be delivered, the lance can correspondingly be charged with a negative polarity in order to associate this positively charged biological material to the tip portion of the lance.

FIG. 1C shows the cell 104 secured by the cell manipulation device 112. The cell can be manipulated, secured, and/or held in position by a variety of mechanisms. It should be noted that any technique, device, or system for manipulating, securing, and/or holding a cell in position is considered to be within the present scope. In one aspect, for example, the cell can be held in position by a suction pipette, as is shown in FIG. 1C (cell manipulation device 112). A slight suction at the end of such a pipette can hold a cell for sufficient time to accomplish a biological material delivery procedure into an organelle of the cell. Additionally, supporting arms or other physically restraining structures can be used to hold the cell in position during the delivery procedure. Various configurations for support structures would be readily apparent to one of ordinary skill in the art once in possession of the present disclosure, and such configurations are considered to be within the present scope.

Furthermore, the cell can be manipulated to reorient and/or reposition the cell in order to orient an organelle into a desired position to facilitate biological material delivery. Such manipulation can simplify the injection procedure by placing the organelle in a position and/or orientation that may be more readily accessible by the lance. This can be accomplished by various techniques, and any such technique of manipulation, repositioning, or reorienting is considered to be within the present scope. In the case of the suction pipette, for example, the suction can be repeatedly applied and released to allow the cell to rotate at the tip of the suction pipette. In other aspects, the cell can be rolled along a support surface to facilitate repositioning.

Once the cell 104 is restrained, the lance 102 can be oriented into a position relative to the cell into which the biological material will be introduced, as is shown in FIG. 1C. The upper right inset of FIG. 1C shows a close up view of the tip portion of the lance 102 having the biological material 110, in this case DNA, associated therewith. The lance 102 can be oriented into a position that is aligned with the organelle 106 of interest, or the lance 102 can be oriented into a position that corresponds to a region of the cell 102 that is expected to contain the organelle at the time of delivery of the biological material. Additionally, it is noted that, while the cell is shown being restrained at the time the lance is being oriented into an alignment position, the cell can be restrained at any time prior to lance alignment. In one aspect, for example, the cell 104 can be restrained prior to the release of the biological material 110 from the biological material delivery device 108, or prior to charging of the lance 102.

As is shown in FIG. 1D, following positioning, the lance 102 penetrates an outer portion of the cell 104 and is directed and inserted into an organelle 106. Thus, in one aspect an organelle 106 of the cell 104 is identified and oriented into a desired position, following which the lance 102 is purposefully directed and inserted into the organelle 106. The cell 104 can be held in position by the cell manipulation device 112 during the injection procedure to minimize movement of the cell. The minimization of movement of the cell can facilitate the insertion of the lance into the organelle, particularly for small organelles such as pronuclei, while also potentially reducing movement-induced damage of the cell.

As is shown in the upper right inset of FIG. 1D, the biological material 110 that is associated with the tip portion of the lance 102 is carried into the organelle 106 along with the lance. In this example, DNA associated with the tip portion of the lance is carried into the pronucleus of the cell along with the lance. In one aspect, the lance is inserted into the cell and into the organelle in a reciprocating motion along an elongate axis of the lance. Such an insertion method may minimize tearing of the cell membrane and internal damage to the cell.

Once the lance 102 is inserted into the organelle 106, the lance 102 is discharged to allow the release of at least a portion of the biological material 110 from the tip portion of the lance 102, thus delivering the biological material 110 to the organelle 106 as is shown in FIG. 1E. The upper right inset of FIG. 1E shows the biological material 110 being dissociated from the lance 102 in the organelle 106. Discharging the lance to release the biological material can be accomplished in a variety of ways. In one aspect, for example, discharging the lance can include decreasing the charge on the lance to a degree that is sufficient to release at least a portion of the biological material from the lance. In another aspect, discharging the lance can include releasing the charge on the lance sufficient to release the biological material or at least a portion of the biological material from the lance. In yet another aspect, discharging the lance can include reversing the polarity of the charge on the lance to release at least a portion of the biological material. Such a reversal charges the lance to a polarity that is opposite from the polarity used to attract the biological material (e.g. the DNA) to the tip portion of the lance. Thus, a positively charged lance can be reversed to a negative charge to cause a negatively charged biological material such as DNA to be released from the surface of the lance. Thus, depending on the manner in which the lance is discharged, from only a portion to substantially all of the biological material associated with the lance can be released.

Following release of the biological material, the lance 102 can be withdrawn from the cell 104 as is shown in FIG. 1F. The biological material 110 delivered to the organelle 106 can remain in the organelle following withdrawal of the lance 102. Once the lance 102 is withdrawn, the cell 104 can be released from the cell manipulation device 112.

The biological material can be a macromolecule or other material that exists outside of the cell that has been preselected for delivery into the cell. Various types of biological materials are contemplated for delivery into a cellular organelle, and any type of biological material that can be electrostatically delivered is considered to be within the present scope. Non-limiting examples of such biological materials can include DNA, cDNA, RNA, siRNA, tRNA, mRNA, microRNA, peptides, synthetic compounds, polymers, dyes, chemical compounds, organic molecules, inorganic molecules, and the like, including combinations thereof. In one aspect, the biological material can include DNA, cDNA, RNA, siRNA, tRNA, mRNA, microRNA, and combinations thereof. In another aspect, the biological material can include DNA and/or cDNA.

Biological material can be delivered to a variety of organelles, and any organelle capable of being targeted and receiving such biological material is considered to be within the present scope. Non-limiting examples of such organelles include nuclei, pronuclei, mitochondria, chloroplasts, vacuoles, endocytic vesicles, lysosomes, and the like. In one specific aspect, the organelle is a pronucleus. Similarly, both prokaryotic and eukaryotic cells are contemplated that can receive biological material, including cells derived from, without limitation, mammals, plants, insects, fish, birds, yeast, fungus, and the like. Additionally, cells can include somatic cells or germ line cells such as, for example, oocytes and zygotes. The enhanced survivability of cells with the present techniques can allow the use of cells and cell types that have previously been difficult to microinject due to their delicate nature.

The various types of organelles contemplated can vary significantly in size, and as such, delivery techniques used to introduce a biological material therein can be varied to accommodate the organelle. For example, organelles such as pronuclei, nuclei, chloroplasts, and vacuoles can be visualized using current optical microscopy. In these cases, a visual determination of the lance tip relative to the organelle can be used to guide the lance into the organelle.

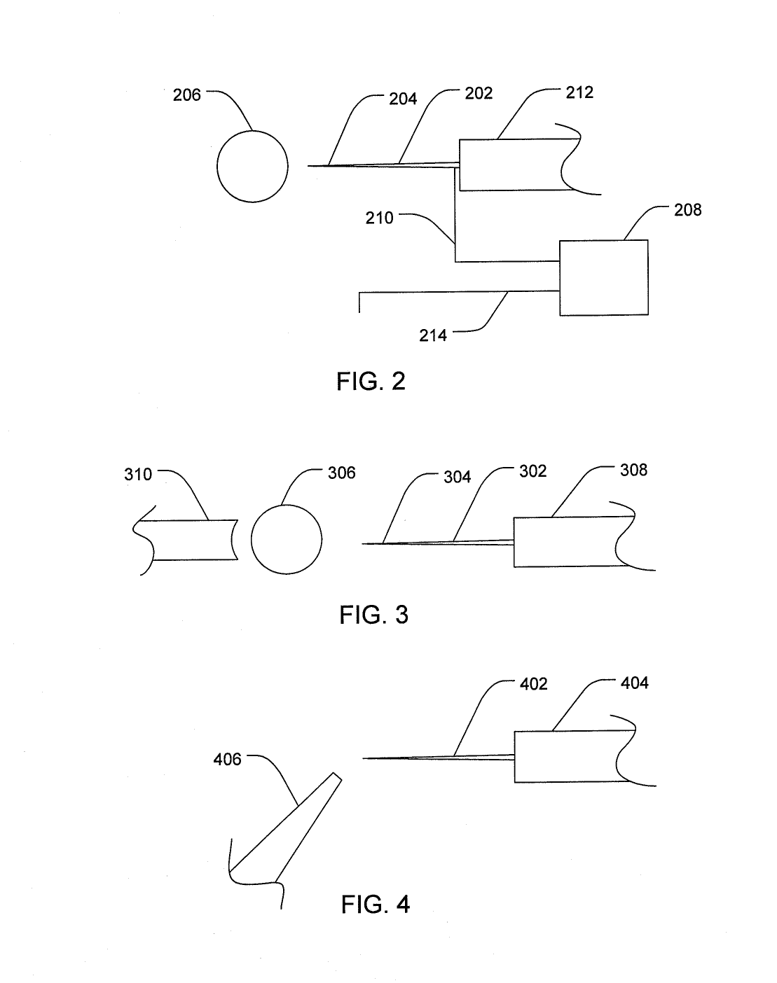

A variety of systems, system configurations, and system components are contemplated for use in delivering a biological material into an organelle of a cell, and any combination of components or configurations is considered to be within the present scope. As is shown in FIG. 2, for example, in one aspect a system for introducing biological material into an organelle of a cell can include a lance 202 having a working portion 204 operable to enter a cell 206, where the working portion has a maximum diameter selected to effectively deliver biological material to an organelle while minimizing damage to the cell. The system can also include a charging system 208 electrically coupleable 210 to the lance 202 and being operable to charge and discharge the lance 202, and a lance manipulation system 212 operable to move the lance 102 into and out of an organelle in a reciprocating motion along an elongate axis of the lance that minimizes damage to the cell 206. The system can also include a return 214 for completing an electrical circuit with the charging system 208.

In another aspect, as is shown in FIG. 3, a system for introducing biological material into an organelle of a cell can include a lance 302 having a working portion 304 operable to enter a cell 306. The system can also include a lance manipulation system 308 operable to move the lance 302 into and out of an organelle in a reciprocating motion along an elongate axis of the lance that minimizes damage to the cell 306. The system can also include a cell manipulation device 310 for holding the cell 306 during a biological material delivery procedure.

In yet another aspect, as is shown in FIG. 4, a system for introducing biological material into an organelle of a cell can include a lance 402 and a lance manipulation system 404 operable to move the lance 402 into and out of an organelle in a reciprocating motion along an elongate axis of the lance that minimizes damage to the cell. The system can also include a biological material delivery device 406 configured to deliver a biological material capable of association with the lance 402. As has been described, the biological material delivery device 406 can be positioned to release biological material in the proximity of a tip portion of the lance 402.

As has been described, a lance can be configured to be inserted into a cellular organelle. As such, the physical configuration of such a lance should be sufficient to allow penetration into an organelle of interest while minimizing damage to the organelle structure. In one aspect, for example, the lance can be a narrow tapered structure having a tip diameter capable of penetrating the organelle while minimizing damage. The physical configuration of the lance can, in some cases, vary depending on the organelle being targeted. For example, a lance used to target an organelle located deep within a cell can be configured with a shape that minimizes the disturbance of the cellular membrane as the lance is inserted through the cell and into the organelle. Such a configuration may include an elongated tapered tip portion having little to no slope at least along the region that is inserted into the cell. Thus, the physical configuration of a given lance can be designed according to the type of cell, the type of organelle, and/or the organelle location within the cell.

Accordingly, any size and/or shape of lance capable of delivering biological material into an organelle is considered to be within the present scope. The size and shape of the lance can also vary depending on the organelle receiving the biological material. The effective diameter of the lance, for example, can be sized to improve the survivability of the cell. It should be noted that the term “diameter” is used loosely, as in some cases the cross section of the lance may not be circular. Limits on the minimum effective diameter of the lance can, in some cases, be a factor of the material from which the lance is made and the manufacturing process used. In one aspect, for example, the lance can have a tip diameter of from about 5 nm to about 3 microns. In another aspect, the lance can have a tip diameter of from about 10 nm to about 2 microns. In another aspect, the lance can have a tip diameter of from about 30 nm to about 1 micron. In a further aspect, the lance can have a tip diameter that is less than or equal to 1 micron. As such, in many cases the tip diameter of the lance can be smaller than the resolving power of current optical microscopes, which is approximately 1 micron. As is noted above, lance tips are contemplated that can have cross sections that are not circular. In such cases, it is intended that the circumference of a circle defined by the tip diameters disclosed above would be substantially the same as an outer circumferential measurement of a non-circular lance tip. One non-limiting example of a non-circular lance tip can have a thickness of about 0.5 to about 2.0 microns and a width of about 17 to about 200 nanometers.

The length of the lance can be variable depending on the design and desired attachment of the lance to the lance manipulation system. Also, the portion of the lance that is contacting and/or passing through a portion of the cell can vary in length depending on the lance design and the depth of the organelle into which the biological material is to be delivered. For example, delivering biological material to an organelle located near the surface of a cell can be accomplished using a shorter lance as compared to delivery to an organelle located deep within the cell. This would not preclude, however, the use of longer lances for delivery into organelles near the cellular surface. For example, a relatively long lance may be used to deliver biological material in an application where only a small portion (e.g., only the tip) of the lance penetrates a cell. It should be noted that the lance length can be tailored to the delivery situation and to the preference of the individual performing the delivery.

Thus the length of the lance can be any length useful for a given delivery operation. For example, in some aspects, the lance can be up to many centimeters in length. In other aspects, the lance can be from a millimeter to a centimeter in length. In another aspect, the lance can be from a micron to a millimeter in length. In one specific aspect, the lance can be from about 2 microns to about 500 microns in length. In another specific aspect, the lance can be from about 2 microns to about 200 microns in length. In yet another specific aspect, the lance can be from about 10 microns to about 75 microns in length. In a further specific aspect, the lance can be from about 40 microns to about 60 microns in length.

Additionally, the shape of the lance, at least through the portion of the lance contacting the cell, can vary depending on the design of the lance and the depth to which the biological material is to be injected into the cell. A high lance taper, for example, may be more disruptive to cellular membranes and internal cellular structures than a low taper. In one aspect, for example, the lance can have a taper of from about 1% to about 10%. In another aspect, the taper can be from about 2% to about 6%. In yet another aspect, the taper can be about 3%. The taper of the lance can also be described in terms of the size of the disruption in the cell membrane following insertion. In one aspect, for example, the approximate diameter of the disrupted area of the cell membrane following lance insertion is from about 10 nanometers to about 8 microns. In another aspect, the approximate diameter of the disrupted area of the cell membrane following lance insertion is from about 2 micron to about 5 microns.

The overall shape and size of the lance can also be designed to take into account various factors, including those involved with the delivery procedure, as well as the materials utilized to make the lance. For example, in one aspect a lance can be designed having sufficient cross sectional strength to allow biological material delivery, while at the same time minimizing the damage done to the cell from the lance's cross sectional area. As another example, the lance can be designed to have a cross sectional area sufficient to minimize damage to the cell, while at the same having sufficient surface area to which biological material can be electrically associated.

Different materials can also affect the design of the size and shape of the lance. Some materials may not hold a charge sufficient to associate the biological material to the lance tip at smaller sizes. In such cases, larger size lances can be used to facilitate a higher charge capacity. It may be difficult in some cases to form particular sizes and shapes of the lance from certain materials. In such cases, the lance size and shape can be designed to the properties of the desired material. For example, a material such as gold may not be capable of supporting the lance tip at very small diameters due to inadequate strength at smaller sizes, or it may not be possible or feasible to create a very small diameter tip with gold. If the use of a gold lance is desired, the lance size and shape can thus be designed with the properties of gold in mind.

As has been described, a charge can be introduced into and held by the lance in order to electrically associate the biological material to the lance. Various lance materials are contemplated for use in constructing the lance, and any material that can be formed into a lance structure and is capable of carrying a charge is considered to be within the present scope. Non-limiting examples of lance materials can include a metal or metal alloy, a conductive glass, a polymeric material, a semiconductor material, and the like, including combinations thereof. Non-limiting examples of metals can include indium, gold, platinum, silver, copper, palladium, tungsten, aluminum, titanium, and the like, including alloys and combinations thereof. Polymeric materials that can be used to construct the needle structure can include any conductive polymer, non-limiting examples of which include polypyrrole doped with dodecyl benzene sulfonate ions, SU-8 polymer with embedded metallic particles, and the like, including combinations thereof. Non-limiting examples of useful semiconductor materials can include germanium, gallium arsenide, and silicon, including various forms of silicon such as amorphous silicon, monocrystalline silicon, polycrystalline silicon, and the like, including combinations thereof. Indium-tin oxide is a material that is also contemplated for use as a lance material. Furthermore, in one aspect the lance can be substantially solid.

Additionally, in some aspects the lance can be a conductive material that is coated on a second material, where the second material provides the physical structure of the lance. Examples can include metal-coated glass or metal-coated quartz lances. The lance can also include a hollow, non-conductive material, such as a glass, where the hollow material is filled with a conductive material. Depending on the design, the lance can be manufactured using various techniques such as wire pulling, chemical etching, MEMs processing, various deposition techniques, and the like.

The charging system can include any system capable of electrically charging, maintaining the charge, and subsequently discharging the lance. Non-limiting examples can include batteries, DC power supplies, photovoltaic cells, static electricity generators, capacitors, and the like. The charging system can include a switch for activation and deactivation, and in some aspects can also include a polarity switch to reverse polarity of the charge on the lance. In one aspect the system may additionally include multiple charging systems, one system for charging the lance with a charge, and another charging system for charging the lance with an opposite polarity charge. In one exemplary scenario, an initially uncharged lance is brought into contact with a sample of a biological material. The biological material can be in water, saline, or any other liquid capable of maintaining biological material. A charge opposite in polarity to the biological material is applied to the lance, thus associating a portion of the biological material with the lance. The lance can then be moved into the organelle of interest, and lance can be discharged, thus releasing the biological material.

The lance can be manipulated by any system or mechanism capable of aligning and moving the lance. Non-limiting examples of lance manipulation systems include mechanical systems, magnetic systems, piezoelectric systems, electrostatic systems, thermo-mechanical systems, pneumatic systems, hydraulic systems, and the like. In one aspect, the lance manipulation system can be one or more micromanipulators. The lance may also be moved manually by a user. For example, a user may push the lance along a track from first location to a second location.

In one aspect, the lance can be moved by the lance manipulation system in a reciprocal motion along an elongate axis of the lance. In other words, the lance can move forward into a cell and backward out of the cell along the same path. By moving along the elongate axis of the lance, the minimum cross sectional area of the lance is driven through cellular structures such as a cell membrane and/or the organelle of interest. This minimal cross sectional exposure can limit the cellular disruption, and thus potentially increasing the success of the biological material delivery procedure.

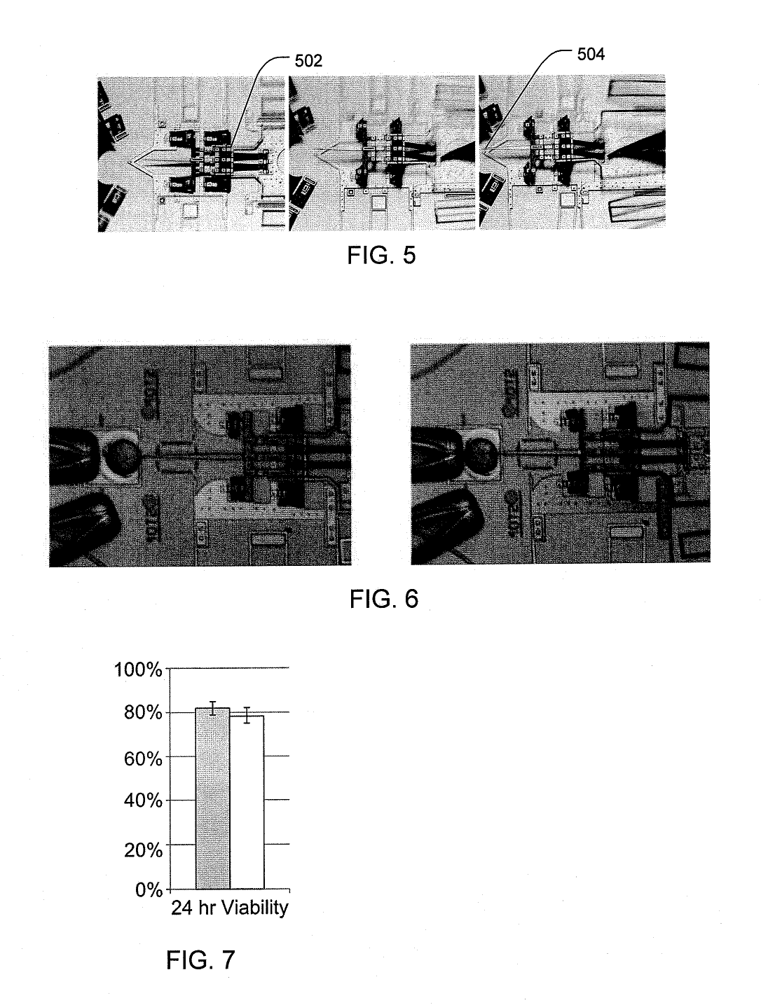

In one exemplary aspect, the lance manipulation system can exhibit a two-stage metamorphic motion, as is shown in FIG. 5. Before actuation, as is shown in the left image of FIG. 5, the lance manipulation system 502 lies in a planar configuration with two polycrystalline silicon layers (e.g. 2.0 μm and 1.5 μm thick) parallel to a fabrication substrate. When actuated as is shown in the middle image, a parallel-guiding, change-point, six-bar mechanism rises from its fabricated position to a final height of about 45 μm, maintaining the lance parallel to the lance manipulation system substrate, while moving about 28 μm horizontally. With continued actuation as is shown in the right image, the tip of the lance 504 moves forward 70 μm with the lance at a fixed height parallel to the substrate by deflecting the compliant folded beam suspension. Throughout the lance manipulation system's 45 μm vertical displacement and 98 μm total horizontal displacements, flexible electrical connections can provide a current path from the stationary bond pads to the lance. While it is clear that the above embodiment is merely exemplary, it should also be noted that the dimensions given for height, displacement, thickness, etc. are also exemplary, and similar embodiments having different dimensions are also contemplated.

Further exemplary details regarding lances, charging systems, lance manipulation systems, and cellular restraining systems can be found in U.S. patent application Ser. No. 12/668,369, filed Sep. 2, 2010; U.S. patent application Ser. No. 12/816,183, filed Jun. 15, 2010; and U.S. Patent Application Ser. No. 61/380,612, filed Sep. 7, 2010, each of which is incorporated herein by reference.

It should be noted that the design of a system for delivering biological material into an organelle of a cell can vary due to the interdependencies of various system parameters. Combinations of features can thus influence other features, both in terms of system design and in terms of system use. Features can thus be mixed and matched to create a delivery system for a given purpose or desirable performance. For example, the materials and configuration chosen for the lance may have properties allowing a greater or lesser charge capacity, thus influencing the voltage, current, and electrical timing of the charging and discharging. A smaller tip diameter can more effectively enter an organelle with potentially less damage, but may have a smaller surface area for the association of biological material. The association capacity of the lance for biological material can thus be increased, for example, by utilizing lance materials capable of holding a higher relative charge, or by utilizing a non-circular shape for the lance tip that increases surface area while minimizing the penetration damage of the lance. Thus, if a particular feature is desired for a lance, other features can be varied to accommodate such a design. As such, it should be understood that the various details described herein should not be seen as limiting, particularly those involving dimensions or values. It is contemplated that a wide variety of design choices are possible, and each are considered to be within the present scope.

Various delivery procedures for introducing biological material into an organelle are contemplated. As one example, DNA can be introduced into a zygote in order to transfect the zygote with the DNA. By introducing the DNA into the zygote's pronucleus before the first mitotic division is complete, integration of the DNA into the zygote's genome can be achieved. In one aspect, such a method can include bringing into proximity a lance and a preselected DNA material outside of the zygote and charging the lance with a polarity and a charge sufficient to electrically associate the DNA material with a tip portion of the lance. The zygote can be repositioned to orient the pronucleus into a desired position for the injection of the DNA. The lance can then penetrate an outer portion of the zygote and be directed and inserted into the pronucleus. Following insertion, the lance can be discharged to release at least a portion of the DNA material into the pronucleus, and the lance can be withdrawn from the zygote.

### EXAMPLES

The following examples are provided to promote a more clear understanding of certain embodiments of the present invention, and are in no way meant as a limitation thereon.

### Example 1

### Transgene Preparation

Nanoinjections are performed using either an enhanced green fluorescent protein transgene with a ubiquitously expressing chicken β-actin promoter (CAG-EGFP, 3018 bp) or a red fluorescent protein (RFP) monomer transgene with the same promoter (CAG-RFPm, 2976 bp). The plasmid pCAG-GFP (Addgene plasmid 11150) is digested using HindIII, ApaL1, and Spe1, and the resulting 3018 by transgene is isolated using low melting temperature agarose gel electrophoresis and purified with Qiagen QIAEX II kit. For RFP studies, the EGFP is removed from the pCAG-GFP plasmid and replaced with an RFP monomer from pDSRedmonomerN1 (ClonTech plasmid 632465). The same restriction endonucleases for digestion of the pCAG-GFP plasmid are used for the CAG-RFP transgene with a resulting product of 2976 bp. Transgene extracted from agarose is quantified by spectrophotometry and prepared in a PBS solution at 10-15 ng/μl for nanoinjection. For microinjection, the transgene is diluted to a concentration of 3 ng/μl in low (0.1M) EDTA TE (pH 7.4) on days 1 and 2, and diluted to a concentration of 2 ng/μl on days 3 and 4.

### Example 2

### Mouse Care and Embryo Culture

For in vitro viability studies, zygotes are harvested from superovulated, outbred CD1 female mice crossed with CD1 male mice 0.5 days post coitus (Charles River Laboratories, Boston, Mass.). CD1 females are treated with 5 units pregnant mare serum gonadotropin (PMS) (EMD Chemicals Cat #367222) at 3 hrs. prior to the dark cycle, then two days later treated with 5 units human chorionic gonadotropin (hCG) (EMD Chemicals, cat #869031) at 5 hours prior to the dark cycle, and set with a fertile CD1 male for breeding. Donor embryos are obtained the following morning (18 hours after hCG injection) from females with a vaginal plug by dissection of cumulus mass from the oviducts. Zygotes are obtained after 2 minutes of suspension of the mass in PBS with 10 mg/ml polyvinylpyrrolidone and 330 units/ml hyaluronidase (Worthington Biochemicals, Lakewood, N.J.). Zygotes are rinsed in M2 medium (Millipore, Billerica, Mass.), then rinsed in PBS, then maintained in a drop of KSOM medium (Millipore) under silicone oil (Sigma Cat #85414) in an incubator at 37° C. and 5% CO₂ before and after nanoinjection. For in vivo studies, embryos are similarly harvested from C7B1/6J×CBA/J F1 mice and cultured in M16 media. After overnight culture of injected embryos, experienced technicians count and transfer the two-cell embryos into 0.5 day pseudo-pregnant C57B1/6J×CBA/J F1 females.

### Example 3

### Zygote Transfection

The introduction of a biological material such as DNA into an organelle using a charged lance is hereafter referred to as nanoinjection. Nanoinjection is performed using a lance manipulation system similar to that shown in FIG. 5. Injections occur in L5-2 ml of room temperature phosphate buffered saline (PBS). With the lance manipulation system elevated to its full height, a positive charge is applied to the lance. A syringe pump expels a solution of DNA (˜0.125 μl at 10-15 ng/μl) from a stationary glass micropipette toward the tip portion of the lance. The negatively charged DNA molecules accumulate on the positively charged lance, and the zygote is oriented and placed in front of the lance using a glass suction micropipette. Once in position, the lance is advanced by a micromanipulator through the zona pellucida, the cell membrane, pronuclear membrane, and into the pronucleus. With the lance in the pronucleus, a negative charge is applied to the lance, thus releasing the accumulated DNA. After incubation in the pronucleus (e.g. ˜10 seconds), the lance is withdrawn. The process is then repeated for each zygote in the experiment. Injected zygotes are returned to KSOM medium under oil and incubated at 37° C. and 5% CO₂. FIG. 6 shows optical images of an exemplary DNA delivery experiment into the pronucleus of a zygote.

### Example 4

### Embry Viability Study

Zygotes are harvested from super-ovulated, outbred CD-1 mouse females, and are either placed directly into culture or injected with DNA. Nanoinjections following the protocol outlined above are performed using either an enhanced green fluorescent protein transgene with a ubiquitously expressing chicken β-actin promoter (CAG-EGFP, 3018 bp) or a red fluorescent protein monomer transgene with the same promoter (CAG-RFPm, 2976 bp). Embryos are imaged after 24 hours, and the rate of progression to two-cell embryos are recorded. FIG. 7 shows the proportions of untreated and nanoinjected zygotes developing to the two-cell stage. Out of 713 untreated zygotes, 559 developed to the two-cell stage (shaded bar). Out of 363 zygotes that are nanoinjected, 299 developed to the two-cell stage (unshaded bar). There is no statistically significant difference in the two-cell development rates for untreated and nanoinjected zygotes. These in vitro results demonstrate that the injection conditions (insertion of an electrically charged, DNA coated, silicon lance into a zygote) do not significantly decrease the zygote's viability. Additionally, these results suggest that the injection conditions (i.e. room temperature PBS) do not have a significant effect on the viability and two-cell stage development of embryos.

### Example 5

### Gestational Viability and Transgene Integration

To demonstrate live pup births and transgene integration following pronuclear nanoinjection, side-by-side nanoinjection and microinjection of CAG-EGFP can be performed and compared. This experiment demonstrates that the coupling of electrical accumulation and release of DNA with precise motion of a MEMS device can deliver DNA to the pronucleus of a mammalian zygote results in gene integration and expression. This experiment also can quantify cell survival, pup birth, gene integration, and gene expression rates for the nanoinjection process as compared to microinjection. As an established method of transgenesis, microinjection can serve both as a positive control for transgene integration and expression, and as a baseline for comparing direct, fluidic gene injection and direct electro-physical gene injection.

Zygotes are harvested from super-ovulated C57BL/6J×CBA/J F1 mouse females and divided between one nanoinjection technician and two microinjection technicians at an experienced transgenic mouse facility (Transgenic and Gene Targeting Mouse Core at the University of Utah). The nanoinjections follow the protocol outlined in Example 3, and the microinjections follow standard procedures for microinjection into a single pronucleus. Injected zygotes are cultured overnight and two-cell embryos are counted and transferred into pseudo-pregnant females by the microinjection technicians. After the pups' birth and weaning, genotypic data is collected by polymerase chain reaction (PCR) of tail snips. The PCR results product is verified by sequencing the PCR products. Transgene expression data is collected by flow cytometry of blood, peritoneal exudates, homogenized thigh muscle, homogenized gut, and homogenized brain.

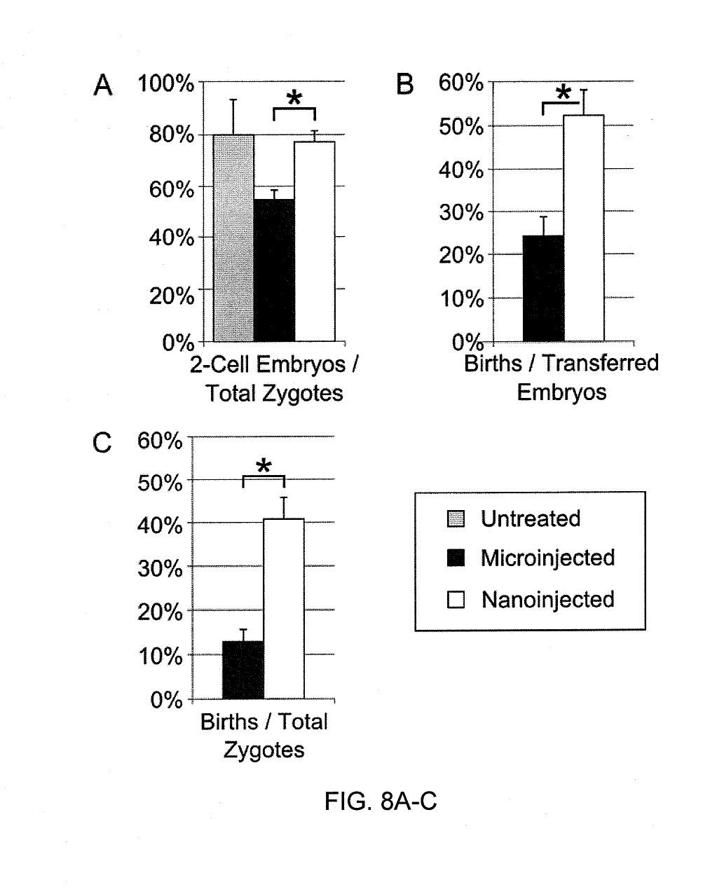

The microinjection technicians culture all the injected zygotes overnight, count the resulting two-cell embryos (FIG. 8A), and then transfer healthy embryos into pseudo-pregnant females. The microinjection technicians also culture a small number of untreated zygotes overnight during the third replicate to estimate the ability of the as-harvested embryos to reach two-cell stage (FIG. 8A). Statistically significant differences (p<0.001) are marked with an asterisk.

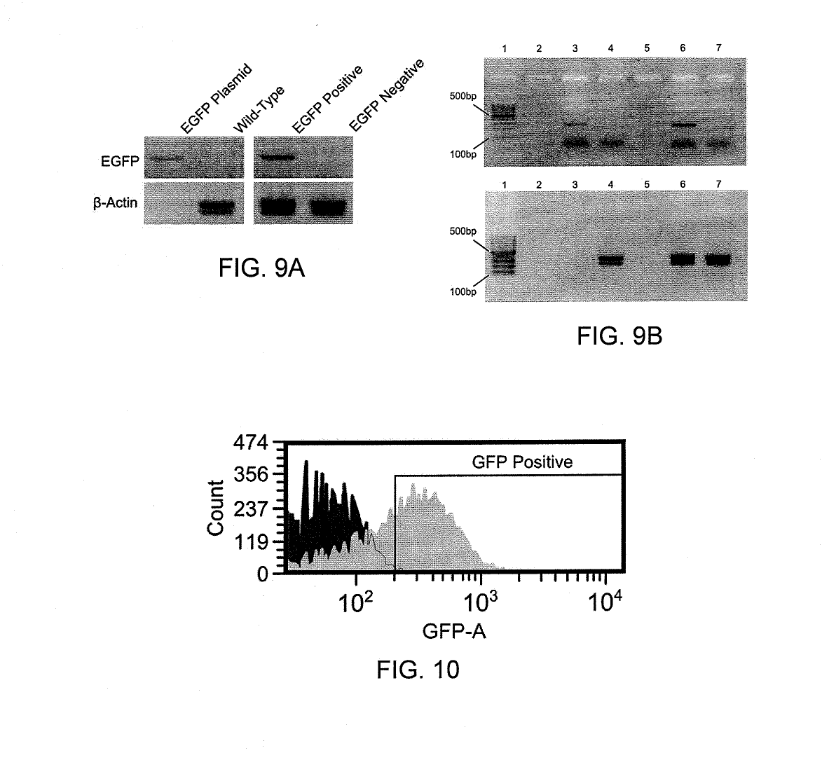

After birth of the mouse pups (FIGS. 8B, C) and weaning, the pups are tested for transgene integration through polymerase chain reaction (PCR) of DNA from tail snips (FIGS. 9A-B). FIG. 9A shows PCR results for EGFP integration occurring in pups. PCR for β-actin serves as a quality control for DNA extracted from tail snips. FIG. 9B shows gels containing PCR samples for genotyping mice. Transgene integration is determined by PCR of DNA samples from pups produced by nanoinjected and microinjected embryos. The upper gel (A) shows PCR of samples to detect GFP and the lower gel (B) shows PCR of samples to detect β-actin to serve as a DNA control. Lanes on the images shown include (1) ladder, (2) blank, (3) EGFP plasmid as a GFP positive control and β-actin negative control, (4) wild type C57B1/6J×CBA/J F1 as a GFP negative control and β-actin positive control, (5) blank, (6) GFP integration positive mouse, and (7) GFP integration negative mouse.

Additionally, DNA samples from integration positive pups and a WT control mouse are analyzed via Southern blot to determine the pattern of transgene integration. Transgene expression data are collected by flow cytometry of blood (FIG. 10), peritoneal exudates, homogenized thigh muscle, homogenized gut, and homogenized brain. FIG. 10 shows flow cytometry of blood samples used to determine whether the integrated transgene could express EGFP. A GFP negative sample is shown in the darker shade on the left and a GFP positive sample is shown in the lighter shade on the right. Both nanoinjection and microinjection produce pups with integrated transgenic DNA during each of the four experimental replicates.

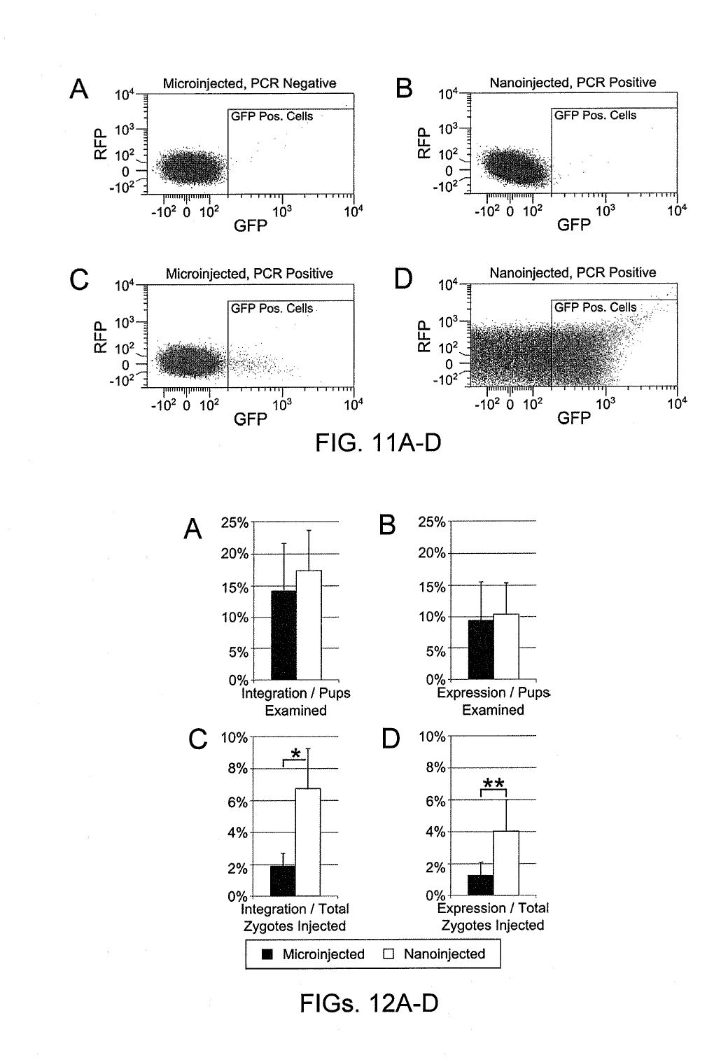

Nanoinjection and microinjection both produce transgenic pups exhibiting silent integration (i.e. PCR positive with no expression), chimeric expression, and full expression. Mice are considered to have full expression if they exhibit high transgene expression in at least two tissues, and are PCR positive. Mice are considered to have chimeric expression if they exhibit high transgene expression in only one tissue. Examples of EGFP expression in blood samples from nanoinjection and microinjection mice are shown in FIGS. 11A-D. FIGS. 11A-D show representative flow cytometry GFP vs. RFP scatter plots of blood samples from pups born from nanoinjected and microinjected zygotes. Each point in the section marked “GFP Pos. Cells” represents a GFP positive cell. In FIG. 11A, a PCR negative and flow cytometry negative microinjection mouse is shown. In FIG. 11B, a PCR positive and flow cytometry negative nanoinjection mouse is shown, demonstrating silent integration of the transgene. In FIG. 11C, a PCR positive and flow cytometry positive microinjection mouse exhibiting low transgene expression is shown. In FIG. 11D, a PCR positive and flow cytometry positive nanoinjection mouse exhibiting high transgene expression is shown. Expression of the nanoinjected transgene indicates that transgene copies are delivered into the pronucleus of mouse zygotes. More specifically, successful nanoinjection of DNA into the pronucleus indicates that DNA electrically accumulates on the lance surface, remains associated with the lance during penetration into the pronucleus, and is electrically released from the lance within the pronucleus.

In examining the viability data, no significant difference is found between the rate of two-cell embryo development for untreated and nanoinjected embryos (FIG. 8A). However, the two-cell development rate is 1.4 times higher for nanoinjected embryos than for microinjected embryos. Additionally, the odds ratio for 2×2 contingency tables indicates that the odds an injected zygote will develop to the two-cell stage is 2.9 times higher for nanoinjection than microinjection (FIG. 8A). When comparing the gestational success of healthy two-cell embryos transferred into surrogate female mice, the percentage of transferred embryos developing into pups is 2.2 times higher for nanoinjected embryos than microinjected embryos (FIG. 8B). As calculated by the odds ratio, the odds of gestational success for each embryo transferred are 3.5 times higher in nanoinjected embryos than in microinjected embryos irrespective of the in vitro viability rate observed between injection and the two-cell stage. Factoring in both the initial embryo survival in overnight culture and the gestational viability, the overall viability of nanoinjected embryos is 3.2 times higher than microinjected embryos. The odds ratio indicates that the odds an injected zygote will develop into a pup are 4.2 times higher with nanoinjection than microinjection (FIG. 8C).

Comparing transgene integration rates in nanoinjected pups with microinjected pups, the rate of transgene integration is not significantly different between the two processes (FIG. 12A-B). Southern blot data confirms that multiple insertion sites and single or multiple integrations can occur through either nanoinjection or microinjection delivery of the transgene. When comparing the percentage of pups with transgene integration or expression, the integration rate is not statistically different between microinjection (integration 10/81, expression 6/81) and nanoinjection (integration 23/140, expression 13/140). Similarity between integration and expression rates among the pups born is not surprising, because both processes rely on the same mechanisms of random gene integration once sufficient transgene copies are delivered to the zygote's pronucleus. The differences between the two injection processes are apparent when taking into account the influence of survival rates on the overall integration and expression success rates (FIG. 12C-D). When compared to the total number of injections performed, nanoinjection results in a significantly higher percentage of integrated (23/371) as well as expressing (13/371) transgenic mice than microinjection (integration 10/642, expression 6/642). Plotted confidence intervals in FIGS. 12A-D are Agresti-Coull 95% confidence intervals for binomial proportions (*The difference between the indicated proportions is statistically significant (p<0.001). **The difference between the indicated proportions is statistically significant (p<0.01).) The higher viability of nanoinjected embryos led to nanoinjection having 4.0 times higher overall transgene integration rates per injected zygote (FIG. 6C). As calculated by the odds ratio, the odds of an injected embryo developing into a pup carrying the transgene are 4.2 times higher for nanoinjection than for microinjection. Thus, fewer egg donor females, embryos, pseudopregnant females, and injection procedures are required to generate each transgenic mouse using nanoinjection.

### Example 6

### Polymerase Chain Reaction

DNA is extracted from tail biopsies through overnight proteinase K digestion and isopropanol precipitation. To ensure DNA quality, each sample is assayed for the mouse β-actin gene using the forward primer 5′-GTGGGCCGCTCTAGGCACCA-3′ and the reverse primer 5′-CGGTTGGCCTTAGGGTTCAGGG-3′ that yields a 244 bp product (see FIG. S2). The presence of the EGFP transgene is assayed using the forward primer 5′-TGCCCGAAGGCTACGTCC-3′ and reverse primer 5′-GCACGCTGCCGTCCTCG-3′ that yields a 267 bp product (see FIG. 9B).

### Example 7

### Southern Blot Analysis

DNA samples from PCR positive pups and a WT control mouse were submitted to TransViragen (Research Triangle Park, N.C.) for Southern Blot analysis. Genomic DNA samples are digested with EcoRI, run on agarose gels, transferred to nylon membranes, and hybridized with a 716 bp chemiluminescent probe (forward primer 5′-ATGGTGAGCAAGGGCGAGGA-3′, reverse primer 5′-TTGTACAGCTCGTCCATCCG-3′).

### Example 8

### Flow Cytometry

Blood samples obtained from weaned pups are diluted in PBS containing 100 units/ml heparin, and peritoneal exudates are obtained by injecting 5 ml of Hanks balanced salt solution (2-3 ml for smaller pups) into the peritoneal cavity. Thigh muscle, brain, and gut tissue samples are homogenized in 2 ml of Hanks, and passed through a 70 μm filter. All samples are stored on ice prior to flow cytometry. Flow cytometry analysis is performed with a BD Biosciences FACSCanto cytometer. Flow data is analyzed using Diva software (BD Biosciences) and Summit software (Dako-Cytomation). Example flow cytometry results are shown in FIGS. 11A-D.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method for introducing biological material into an organelle of a cell, comprising:
bringing into proximity outside of a cell a lance and a preselected biological material;
charging the lance with a polarity and a charge sufficient to electrically associate the preselected biological material with a tip portion of the lance;
penetrating an outer portion of the cell with the lance and directing and inserting the lance into an organelle of the cell;
discharging the lance to release at least a portion of the biological material into the organelle; and
withdrawing the lance from the cell.

2. The method of claim 1, further comprising manipulating the cell to orient the organelle into a desired position prior to penetrating the outer portion of the cell with the lance.

3. The method of claim 1, further comprising securing the cell prior to penetrating the outer portion and releasing the cell following withdrawing the lance from the cell.

4. The method of claim 1, wherein the organelle includes a member selected from the group consisting of a nucleus, a pronucleus, a mitochondria, a chloroplast, a vacuole, an endocytic vesicle, and a lysosome.

5. The method of claim 1, wherein the organelle is a pronucleus.

6. The method of claim 1, wherein inserting and withdrawing the lance is performed with a reciprocating motion along an elongate axis of the lance.

7. The method of claim 1, wherein discharging the lance includes decreasing the charge sufficient to release at least a portion of the biological material from the tip portion of the lance.

8. The method of claim 1, wherein discharging the lance includes reversing the polarity of the charge on the lance to release at least a portion of the biological material from the tip portion of the lance.

9. The method of claim 8, wherein discharging the lance further includes increasing the charge to a degree sufficient to release substantially all of the associated biological material from the tip portion of the lance.

10. The method of claim 1, wherein the biological material includes a member selected from the group consisting of DNA, RNA, peptides, polymers, organic molecules, inorganic molecules, ions, and combinations thereof.

11. The method of claim 1, wherein the biological material includes DNA.

12. A method for transfecting a zygote with a biological material, comprising:
bringing into proximity a lance and a preselected DNA material outside of a zygote;
charging the lance with a polarity and a charge sufficient to electrically associate the preselected DNA material with a tip portion of the lance;
manipulating the zygote to orient a pronucleus of the zygote into a desired position;
penetrating an outer portion of the zygote with the lance and directing and inserting the lance into the pronucleus;
discharging the lance to release at least a portion of the DNA material into the pronucleus; and
withdrawing the lance from the zygote.

13. A system for introducing biological material into an organelle of a cell, comprising:
a lance having a working portion operable to enter a cell, the working portion having a maximum diameter selected to effectively deliver biological material to an organelle while minimizing damage to the cell;
a charging system electrically coupleable to the lance and being operable to charge and discharge the lance; and
a lance manipulation system operable to move the lance into and out of an organelle in a reciprocating motion along an elongate axis of the lance that minimizes damage to the cell.

14. The system of claim 13, further comprising a biological material delivery device configured to deliver a biological material capable of association with the lance.

15. The system of claim 14, wherein the biological material delivery device is positioned to deliver the biological material to contact the lance.

16. The system of claim 13, further comprising a preselected biological material sample electrically associated with a tip portion of the lance.

17. The system of claim 13, further comprising a single cell positioned to receive the lance upon operation of the lance manipulation system.

18. The system of claim 17, wherein the single cell is a zygote.
